(19) United States
(12) Patent Application Publication
(10) Pub. No.: US 2022/0015783 A1
(43) Pub. Date: Jan. 20, 2022

(54) NOVEL ENHANCED ENDOVASCULAR MECHANICAL THROMBECTOMY DEVICES AND TECHNIQUES

(71) Applicant: LAMBERT RADIOLOGY MEDICAL GROUP, Whittier, CA (US)

(72) Inventor: Shao-Pow Lin, Arcadia, CA (US)

(73) Assignee: LAMBERT RADIOLOGY MEDICAL GROUP, Whittier, CA (US)

(21) Appl. No.: 17/387,900

(22) Filed: Jul. 28, 2021

Related U.S. Application Data
(63) Continuation of application No. 16/661,301, filed on Oct. 23, 2019, now abandoned.
(60) Provisional application No. 62/749,744, filed on Oct. 24, 2018.

(51) Int. Cl. A61B 17/22

(52) U.S. Cl. CPC A61B 17/22; A61M 25/0108; A61B 2017/22079

(57) ABSTRACT

A novel variable diameter aspiration catheter (VDAC) device and method for an enhanced endovascular mechanical thrombectomy technique is disclosed. In an aspect, the use of a VDAC allows endovascular mechanical thrombectomy techniques to be performed with “full flow control”.

### RELATED APPLICATIONS

The present application is a continuation of U.S. patent application Ser. No. 16/661,301, filed Oct. 23, 2019, which in turn claims benefit of U.S. Provisional Patent Application Ser. No. 62/749,744, filed Oct. 24, 2018. The contents of the aforementioned applications are incorporated by reference herein.

### FIELD

The present disclosure generally relates to the field of endovascular procedures, and more specifically to endovascular thrombectomy devices for the endovascular procedures within the neurovascular space.

### BACKGROUND

Referring to FIG. 1A, all conventional endovascular mechanical thrombectomy (EVMT) techniques (including Technique #s 1-4 below) for removal of thrombus (clot) in the setting of acute stroke rely on the same basic principles:

a. A guide catheter 100 is navigated to the internal carotid artery (ICA), either via the femoral or radial approach, but typically via the femoral artery. The guide catheter is navigated over a standard guidewire (typically 0.035 inch diameter), after which the guidewire is removed. Some of the guide catheters in use have a balloon at the tip that allows for management of ICA flow during clot manipulation.

b. An intermediate aspiration catheter 200 and/or a microcatheter 300 are then coaxially delivered to the site of the thrombus 50 (clot) over a microguidewire (typically 0.014-0.018 inch diameter). Clot 50 is removed either with a stent-retriever 150 (which is delivered via the microcatheter 300), by direct aspiration into the intermediate aspiration catheter 200, or a combination thereof. This will be described in detail for each of the individual techniques.

Technique #1—Stent-retriever with or without balloon guide.

Referring to FIG. 2A, a conventional technique predominantly utilized in the positive randomized controlled EVMT trials involved use of a stent-retriever 150 (e.g., Solitaire™ revascularization devices by Medtronic vs. Stryker's Trevo® Retriever devices) delivered to the site of occlusion through a microcatheter 300 (not shown). In this technique, the tip of a guide catheter 100 is first navigated over a standard guidewire (typically 0.035 inch diameter) to the ICA. The guidewire is then removed. Next, in a coaxial fashion, the combination of a microcatheter 300 and microguidewire 155 is navigated beyond the clot 50 (typically around the periphery of the clot, i.e., between the clot and inner vessel wall of the MCA). The microguidewire 155 is then removed (leaving both catheters 100 and 300 in place). There is no intermediate aspiration catheter utilized in this technique. The stent-retriever 150 (which consists of a self-expanding stent-like device permanently attached to the tip of a microguidewire) is then advanced through the microcatheter 300, and positioned (still within the microcatheter 300) such that it spans the target clot 50. The microcatheter 300 is then withdrawn, which unsheaths the stent-retriever 150, allowing it to open. As this happens, different types of stent-retrievers 150 interact differently with different types of clot 50, which can range in consistency from soft and friable to firm and rubbery. Ideally, the stent-retriever 150 penetrates through the clot 50 as it opens such that the clot 50 is now either enveloped or otherwise attached to the stent-retriever 150 so it can be removed. With a very firm clot 50, however, the stent-retriever 150 may not penetrate at all, such that removal of the stent-retriever 150 can result in a “rolling” or “dragging” effect that is ultimately effective in dislodging the clot 50, but ineffective in actually retrieving it.

The use of a balloon guide catheter (BGC) 100 (e.g., Flowgate²™ by Stryker or Cello™ by Fuji Systems Corporation) for proximal flow control in the positive randomized controlled trials of EVMT was variable. Sub-analyses of retrieval data suggest that utilization of a balloon guide catheter is associated with higher rates of reperfusion, higher rates of first pass reperfusion, lower rates of distal embolization, and better clinical outcomes (“First pass effect” refers to the improved likelihood of a good patient outcome when the clot 50 is removed on the first attempt.) Although these associations have not been proven in a separate randomized trial, the concept behind this technique is compelling and will be discussed in detail below.

Each component of a typical tri-axial catheter construct for EVMT (see FIG. 1) serves a specific purpose. For example, the stent-retriever 150 is designed to engage the thrombus 50, dislodge it 50 from the affected artery, and ideally remove it 50 in one piece. The BGC 100, when utilized, functions as a back-up system to the stent-retriever 150 by managing the flow in the ICA and Circle of Willis during clot manipulation.

There are two components to what we refer to as “full flow control”:

a) Proximal flow arrest, i.e., prevention of antegrade flow, which is achieved with balloon inflation in the internal carotid artery (ICA).

b) Flow reversal, which is achieved with the application of suction on the BGC during thrombectomy (also see FIG. 2A).

Referring to FIGS. 1B-1C, it is very important to point out that when there is only flow arrest without flow reversal (i.e. balloon inflation with no or ineffective suction), the direction of flow in the middle cerebral artery (MCA) after a clot is dislodged is variable, depending on the quality of collateral circulation (FIG. 1B). Specifically, it depends on the relative robustness of competing inputs from the Circle of Willis (antegrade flow) and the pial collateral network (retrograde flow). In this situation, any free or fragmented thrombus 50 in the MCA or around the carotid terminus has the potential to migrate out to distal branches. By adding flow reversal to the equation (FIG. 1C), inflow from the Circle of Willis via the anterior and/or posterior communicating arteries is forced down the ICA to the BGC 100, removing all sources of antegrade MCA flow.

It stands to reason that full flow control (i.e. arrest with reversal) during thrombectomy has the following advantages:
c. In the event the stent-retriever 150 causes fragmentation of the thrombus 50, flow reversal prevents distal embolization by forcing any loose clot fragments in the MCA or around the carotid terminus down the ICA to the BGC, through which they are sucked out of the patient.
d. In the event the stent-retriever 150 disengages from the thrombus 50 after the thrombus 50 has been successfully dislodged out of the MCA, reversal of blood flow by the BGC 100 still has the potential to force thrombus 50 down the internal carotid artery and out through the BGC 100.

Technique #2—A Direct Aspiration First Pass Technique (ADAPT)

Referring to FIG. 2B, another conventional technique called “a direct aspiration first pass technique for thrombectomy”, also known as the ADAPT technique, involves navigating an intermediate aspiration catheter 200 (for example, one having an inner diameter (ID) ranging from approximately 0.060 to 0.070 inches) to the proximal face of the thrombus 50 in the MCA (or ICA) over a microcatheter (300; FIG. 1) and within a guide catheter 100 for direct aspiration of the thrombus 50. The intermediate-aspiration catheter 200 is delivered in a co-axial technique as described above with respect to FIG. 1: within a larger bore guide catheter 100 and outside of (over) a microcatheter 150/microguidewire 155 combination. In this technique, the microcatheter 150/microguidewire 155 are removed. Local suction is then applied “directly to the thrombus,” i.e., suction is applied to the intermediate aspiration catheter 200 to directly aspirate the thrombus 50.

The advantages of the ADAPT approach are its simplicity and potential to capture and remove the thrombus 50 in one piece, with little to no mechanical manipulation that could result in fragmentation thereof.

However, in the event of clot fragmentation, particularly when the proximal fragment of the ingested clot is sucked into but not all the way through the intermediate aspiration catheter 200 (known as “corking”), there is no control of the distal aspect of the thrombus 50, which is now free to potentially embolize distally. In other words, the major drawback of the ADAPT technique is that one must count on the intermediate catheter 200 to either: 1) fully engulf and suck out the clot 50, or if that does not occur; 2) engage (suck in) enough of the proximal aspect of the clot 50 (i.e., “cork” the tip of the intermediate aspiration catheter) to remove the entire clot 50 in a single piece as the intermediate aspiration catheter 200 is pulled out. In either case, if the clot 50 breaks, there would be a piece left behind that can undesirably move into a distal branch of the MCA.

In theory, this drawback of the ADAPT technique could be partially mitigated by providing proximal flow arrest with use of a balloon guide catheter. Inflation of a BGC 100 in the ICA would prevent antegrade ICA flow; however true flow reversal with the BGC 100 is not possible due to the large size of the intermediate aspiration catheter 200 within it. That is, the outer diameter (OD) of a typical intermediate aspiration catheter 200 is just small enough to be delivered inside of a guide catheter 100, which typically has an ID of approximately 0.084-0.088 inch, thereby being essentially occlusive. For example, still referring to FIG. 2B, in a typical ADAPT technique, the intermediate aspiration catheter 200 occupies approximately 90% of the BGC cross sectional area, such that suction applied to the BGC would be completely ineffective for ICA flow reversal. In practice, there is typically zero retrograde flow.

Technique #3—Stent-Retriever with Local Aspiration (Solumbra technique)

Referring to FIG. 2C, a hybrid method known as the Solumbra technique utilizes a stent-retriever 150 concomitantly with an intermediate aspiration catheter 200 in the MCA (originally conceived with a combination of a Solitaire stent-retriever with a Penumbra aspiration catheter, hence the name). As with the aforementioned Techniques #1 and 2, the Solumbra technique can be performed either with a guide catheter 100 with balloon tip 110 (i.e. balloon guide catheter as shown in FIGS. 1 and 2A) or a non-balloon guide catheter 100. As demonstrated in FIG. 2C, this technique is typically performed with a non-balloon guide catheter. In the same coaxial fashion as described in FIG. 1, the tip of the guide catheter 100 is first positioned in the ICA, followed by positioning of the distal end of an intermediate aspiration catheter 200 near the proximal end of the target clot 50 (typically within the MCA). Next, the combination of a microcatheter 300 and microguidewire 155 is navigated beyond the clot 50 (typically around the periphery of the clot, i.e., between the clot and inner vessel wall of the MCA).

The microguidewire 155 is then removed (leaving all 3 of the catheters 100, 200, and 300 in place). The stent-retriever 150 (which consists of a self-expanding stent-like device permanently attached to the tip of a microguidewire) is then advanced through the microcatheter 300 and deployed (unsheathed) at the target (clot 50). After the stent-retriever 150 is deployed, practitioners often allow it to dwell for about 3-5 min to integrate into the thrombus 50. Next, the microcatheter 300 can either be withdrawn into the intermediate aspiration catheter 200 or removed entirely therefrom. Oftentimes, practitioners remove the microcatheter 300 entirely, as leaving it only takes up space in the intermediate aspiration catheter 200 and decreases retrograde flow during clot retrieval.

When the microcatheter 300 has been removed (or retracted), this leaves the guide catheter 100, intermediate aspiration catheter 200, and stent-retriever 150 in place. After the thrombus 50 has integrated into the stent-retriever 150, local suction is applied in the MCA (and eventually to the thrombus 50 itself) via the intermediate aspiration catheter 200 as the stent-retriever 150 is retracted into it 200 and thereafter removed.

Both the intermediate aspiration catheter 200 and the guide catheter 100 remain in the same position during removal of the stent-retriever 150.

There are several potential advantages to this approach. Leaving the local aspiration catheter 200 in the MCA expedites any additional stent-retriever 150 passes, as there is no need to re-navigate through the tortuous distal ICA. In addition, as clot 50 is never dragged back across the ICA terminus, the anterior cerebral artery remains protected from unintended embolization.

Lastly, as the local, intermediate aspiration catheter 200 is nearly occlusive in the MCA, this may obviate the need for proximal flow arrest to prevent distal embolization.

The primary disadvantage of the Solumbra approach relates to the size of the thrombus 50 relative to the intermediate aspiration catheter 200's ID. A completely occlusive MCA clot would have to be larger in diameter than the ID of any catheter inside the MCA. As a result, attempting to pull this clot 50 into the local aspiration catheter 200 may actually result in stripping of the margins and creation of smaller fragments. It is also important to point out that clots 50 are not typically trapped inside the stent-retriever 150, rather they are engaged in an eccentric fashion, and hence are not “centered” on the catheter lumen. Particularly if the thrombus 50 is firm, this can result in the thrombus 50 becoming lodged between the stent-retriever 150 and intermediate aspiration catheter 200. This may be a desirable effect if the two devices can be easily removed as a unit (see TRAP technique, next section), but can also undesirably result in the entire stent-retriever/catheter/clot complex becoming wedged in the MCA, requiring excessive force to remove them.

Technique #4. Stent-Retriever with Local Aspiration and Flow Arrest

Referring to FIG. 3, another conventional technique called the Trevo Aspiration Proximal Flow Control (TRAP) technique is designed to combine the advantages of the direct stent-retriever and Solumbra techniques (#1 and #3).

In this instance, a balloon guide catheter 100 is positioned in the ICA. An intermediate aspiration catheter 200 and a microcatheter 300 (not shown in FIG. 3; see FIG. 1) are introduced coaxially over a microguidewire 155. The intermediate aspiration catheter 200 (tip thereof) is positioned proximal to the thrombus 50, and the microcatheter 300 is navigated distal to the thrombus 50 (again, as in FIG. 1). The microguidewire 155 is then exchanged for a stent-retriever 150, in a process that is similar to the Solumbra technique described above.

The stent-retriever 150, while still inside the microcatheter 300, is then positioned such that it spans the clot 50. The microcatheter 300 is then withdrawn, which unsheaths the stent-retriever 150, allowing it to open and engage the clot 50. Local suction is then applied to the intermediate aspiration catheter 200, which is advanced until it captures the proximal aspect of the clot 50 with suction (FIG. 3). In this case, “capture” means making contact with and applying suction force to the clot 50. In theory, just the proximal end of the clot 50 is engaged with suction into the tip of the intermediate aspiration catheter 200. Withdrawal of the intermediate aspiration catheter 200 pulls on the proximal end of the clot 50. Withdrawing the stent-retriever 150 at the same time as a unit simultaneously also pulls on the distal aspect of the clot 50. Removing the stent-retriever 150 and the intermediate aspiration catheter 200 as a single unit, without fully retracting the stent-retriever 150 into the aspiration catheter 200, avoids stripping of the clot 50. The primary advantage of this technique is the decreased likelihood of loss of dislodged clot 50 and clot fragmentation during the retrieval process, as a result of having control of both proximal and distal ends of the thrombus 50. There is evidence that this enhances the first pass effect. Proximal flow arrest using a balloon guide catheter 100 also decreases the risk of distal embolization to some extent. Unfortunately, the intermediate aspiration catheter 200 still precludes true flow reversal, as described above for ADAPT. So in comparison to Technique #1, the advantage gained with the TRAP technique (control of both the proximal and distal ends of the clot 50) comes at the cost of losing “Full flow control”.

### SUMMARY

In an aspect, a thrombectomy catheter according to one or more embodiments of the present disclosure includes a distal tip having a tip inner diameter (ID); a tip outer diameter (OD); a tip distal end; a tip proximal end; a flared end towards the tip distal end having a flared end ID and a flared end OD; and a variable diameter portion towards the tip proximal end having a variable outer diameter (OD), The thrombectomy catheter may additionally include a proximal shaft having a shaft inner diameter (ID); a shaft outer diameter (OD); a shaft proximal end; and a shaft distal end. In an aspect, the shaft distal end is continuous with the tip proximal end.

In an aspect, the distal tip has a length of about 1-4 centimeters.

In an aspect, the length is about 1-3 centimeters.

In an aspect, the flared end ID is about 0.060 to 0.072 inches.

In an aspect, the tip ID is at least about 0.060 inches.

In an aspect, the flared end ID is within a range of 4 F to 6 F.

In an aspect, the tip ID is about 0.068 inches.

In an aspect, the flared end ID is greater than the shaft ID.

In an aspect, the shaft OD is about 0.054 inches or more.

In an aspect, the shaft ID is about 0.044 inches or more.

In an aspect, the variable diameter portion includes a portion proximal end, a portion distal end, and a variable inner diameter (ID) between the portion proximal end and the portion distal end.

In an aspect, the shaft OD is configured to allow true reversal of flow into a guide catheter when the thrombectomy catheter is placed inside the guide catheter.

In an aspect, the shaft OD is between about 0.050 to 0.060 inches.

In an aspect, the proximal shaft prevents collapse under vacuum.

In an aspect, the thrombectomy catheter has a total length of about 132 centimeters.

In an aspect, the flared end is made of an elastomer suitable for medical application.

In an aspect, the flared end OD is about 0.080 inches or less.

In an aspect, the flared end include as marker band.

In an aspect, the proximal shaft is made of an elastomer suitable for medical application.

In an aspect, the proximal shaft further includes an inside layer made of sufficiently lubricious material.

In an aspect, the proximal shaft has a high collapse resistance.

In an aspect, the proximal shaft OD is configured to provide an annular space of about 0.014 inches when the thrombectomy catheter is placed inside a guide catheter.

In an aspect, the proximal shaft further includes an outer layer.

In an aspect, the proximal shaft includes a wall formed of a braid construction.

In an aspect, the flared end is at least partially radiopaque.

### BRIEF DESCRIPTION OF THE DRAWINGS

Various preferred embodiments are described herein with references to the drawings in which merely illustrative views are offered for consideration, whereby:

FIG. 1A is a schematic depiction of a typical tri-axial catheter construct for conventional EVMT techniques;

FIG. 1B is a schematic depiction of flow arrest.

FIG. 1C is a schematic depiction of flow reversal.

FIG. 2A is a schematic depiction of a standard stent-retriever with a balloon guide;

FIG. 2B is a schematic depiction of an ADAPT technique;

FIG. 2C is a schematic depiction of a Solumbra technique;

FIG. 3 is a schematic depiction of a TRAP technique;

FIG. 4 shows a schematic illustration of a Variable Diameter Aspiration Catheter (VDAC) in accordance with an embodiment of the present disclosure;

FIG. 5 is a schematic showing an example of a TRAP technique with VDAC in accordance with an embodiment of the present disclosure.

FIGS. 6-8 show schematic illustrations of Variable Diameter Aspiration Catheter (VDAC)s in accordance with some other embodiments of the present disclosure.

FIG. 9 is a schematic of a typical balloon guide catheter shown as attached to a standard proximal hub;

FIG. 10 is a schematic of a typical intermediate aspiration catheter shown as attached to a standard proximal hub;

FIG. 11 is a schematic of a typical delivery microcatheter shown as attached to a standard proximal hub; and

FIG. 12 is a schematic of a variable diameter aspiration catheter in accordance with an embodiment of the present disclosure, shown as attached to a standard proximal hub.

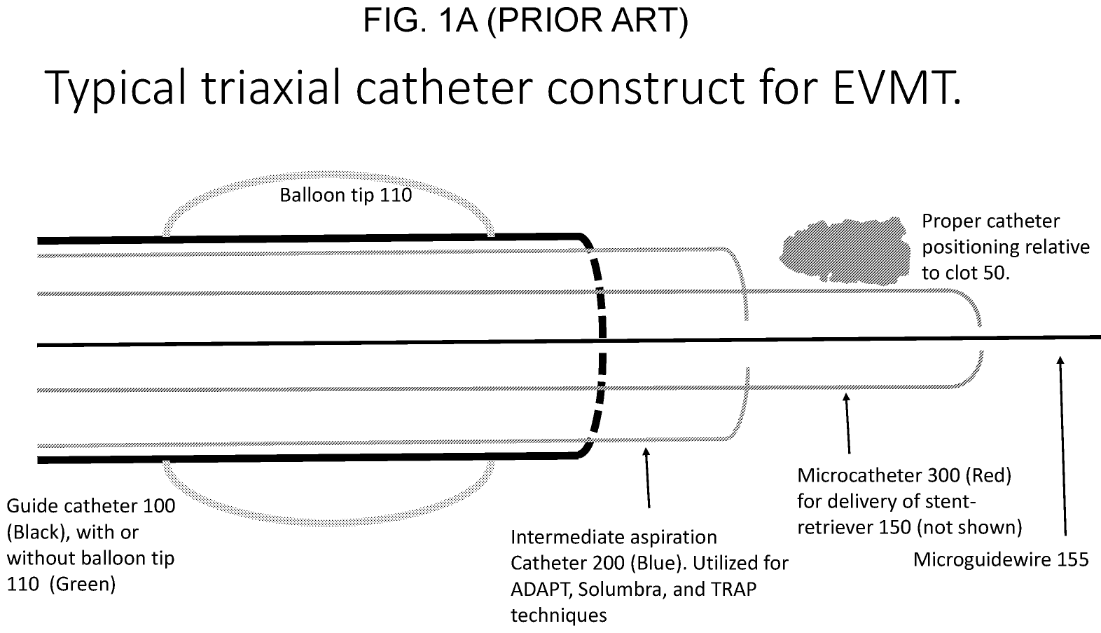

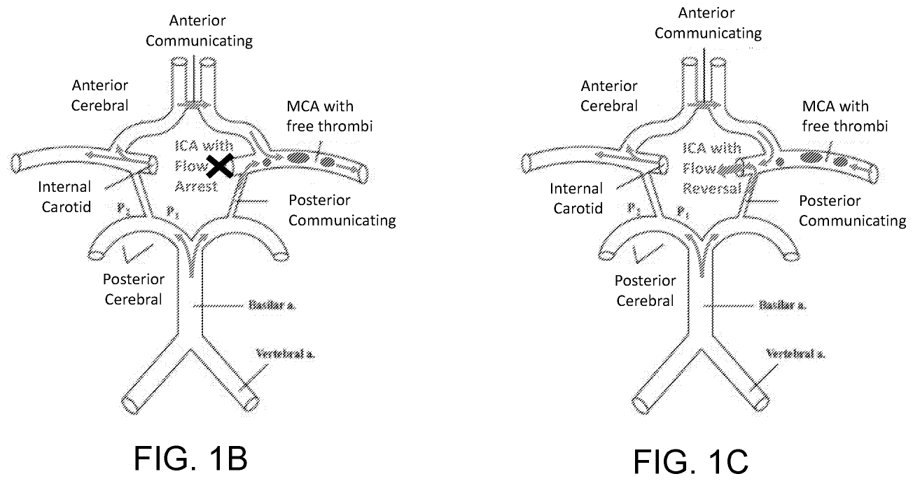

Corresponding reference characters shall later be inserted to indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION

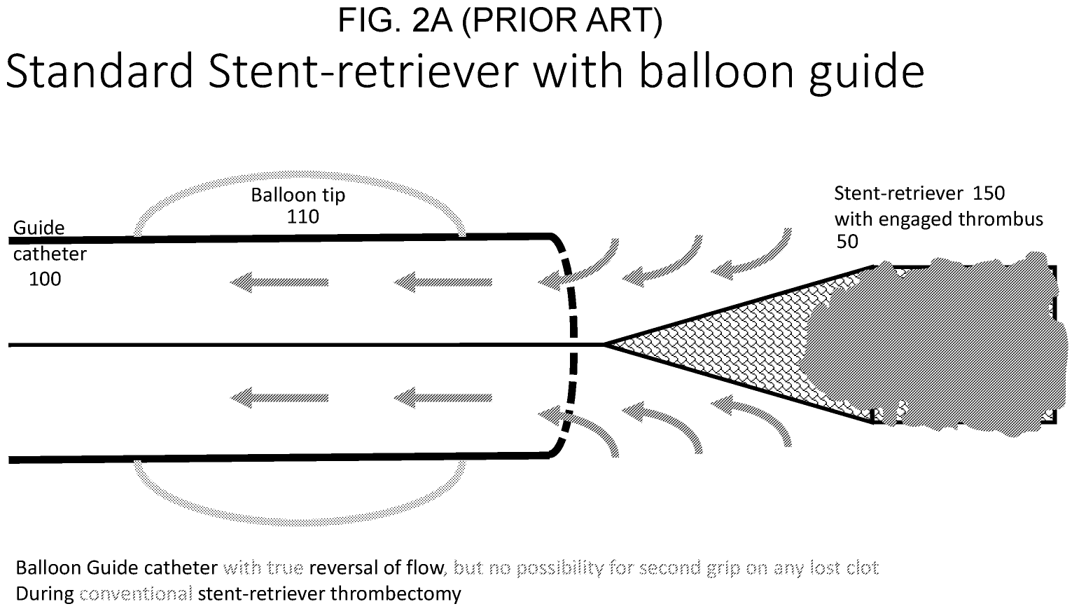

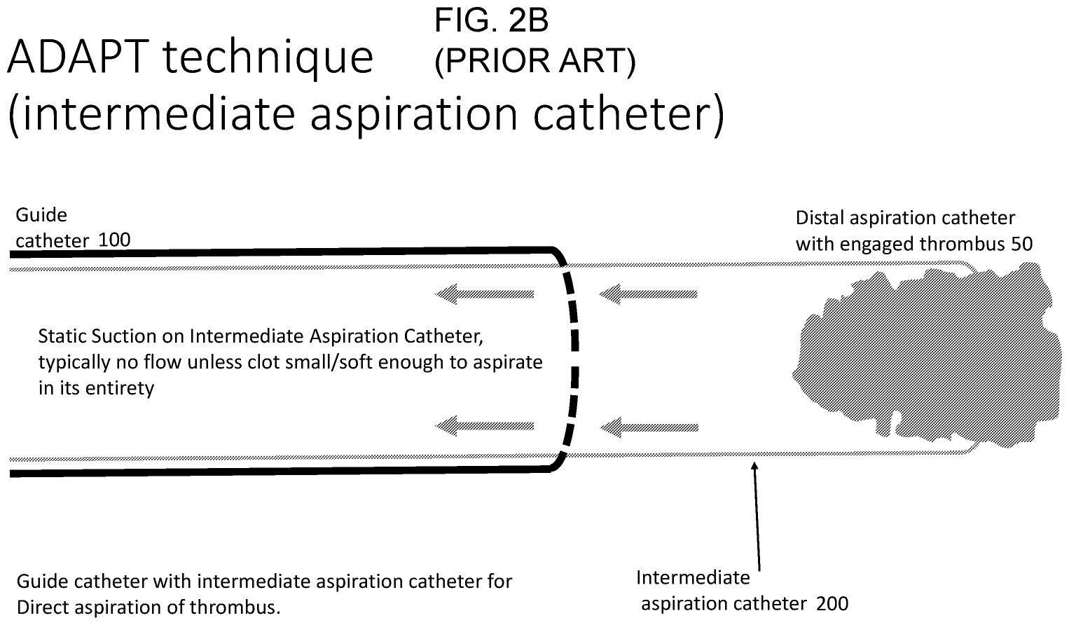

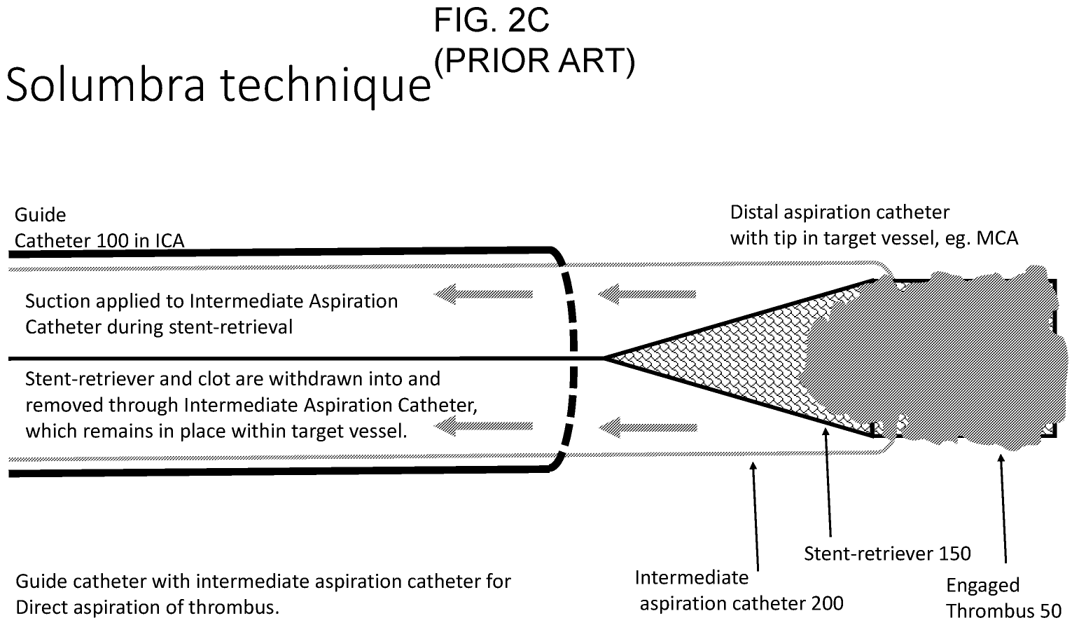

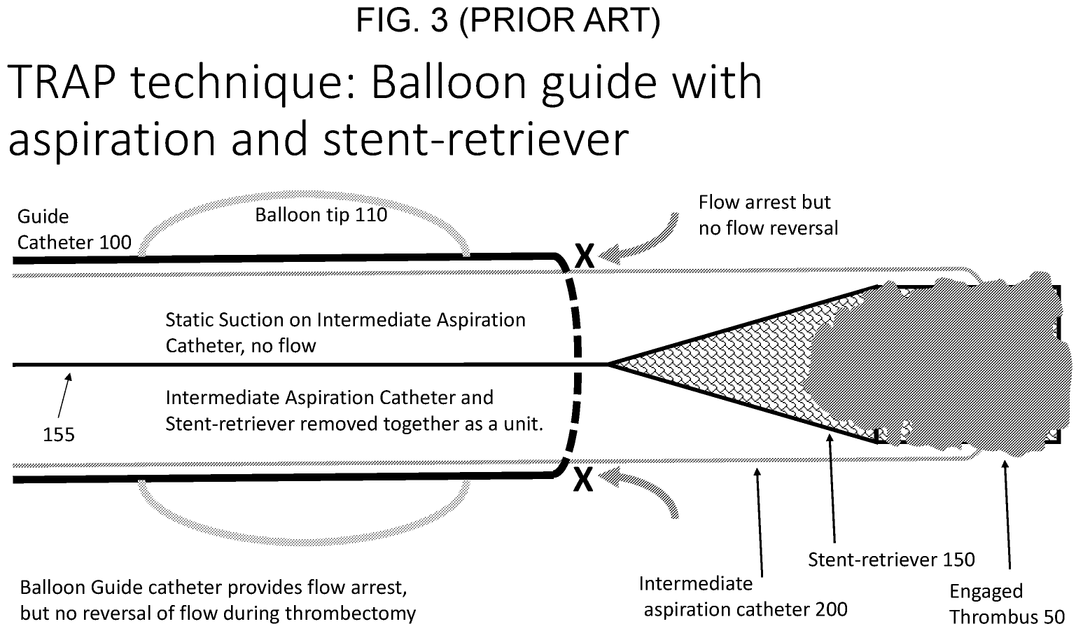

There is substantial evidence that “full flow control” with a BGC during thrombectomy improves first pass success rates. A competing Technique #1 (BGC with stent-retriever) can be compatible with “full flow control” with a BGC; however, it does not allow for second grip on any lost clot (FIG. 2A). Moreover, competing Techniques #2 (ADAPT), #3 (Solumbra), and #4 (TRAP) are not compatible with “full flow control” as they stand. Some of these alternative techniques include aspects that can be improved. In particular, the TRAP technique, which simultaneously engages the clot with BOTH a stent-retriever and intermediate aspiration catheter, has been shown to improve first pass clearance rates in and of itself. The present disclosure uniquely leverages this aspect of the TRAP technique with novel variable diameter aspiration catheter (VDAC) devices for an enhanced endovascular mechanical thrombectomy technique. Specifically, the use of a VDAC instead of a typical intermediate aspiration catheter allows the TRAP technique to be performed with “full flow control”.

Variable Diameter Aspiration Catheter (VDAC)

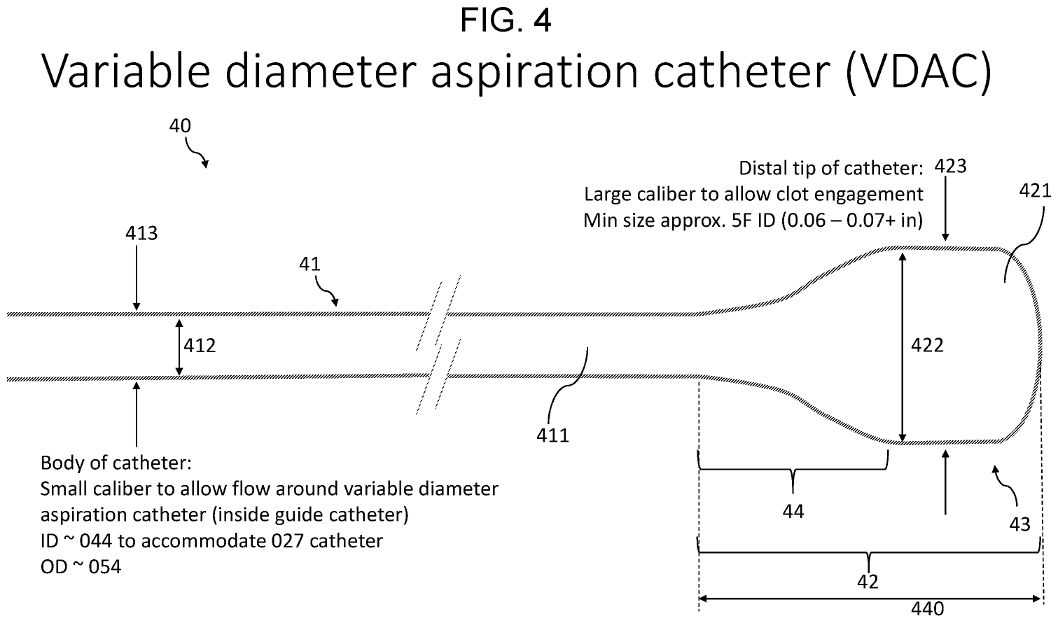

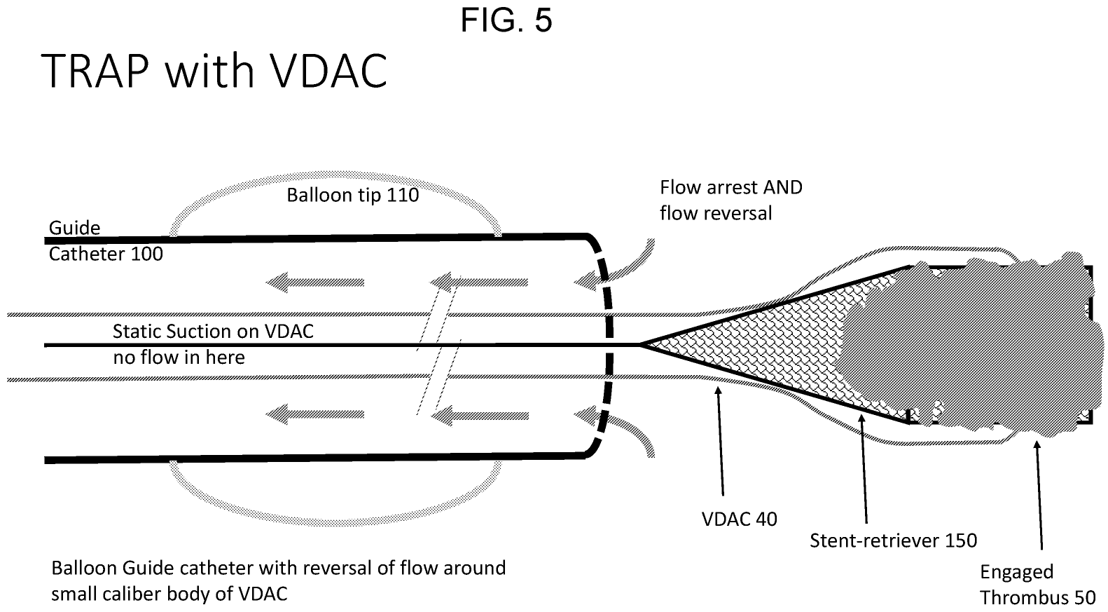

Referring to FIGS. 4 and 5, a variable diameter intermediate aspiration catheter 40 would allow the TRAP technique to be performed with true flow reversal.

In an aspect, the catheter 40 according to an embodiment of the present disclosure has a distal tip 42 having a length 440 of about 1-4 centimeters (cm), and preferably about 1-3 cm, which includes a flared end 43. In an aspect, the flared end 43 is flared to the same or similar inner diameter (ID) 422 as a typical intermediate aspiration catheter (about 0.060 to 0.072 inches). In an aspect, the ID 422 of the distal tip 42 of the VDAC 40 according to an embodiment of the present disclosure is approximately 0.060 inches at a minimum. In an aspect, the flared end 42 may be flared to the same or similar inner diameter 422 ranging from 4 F to 6 F, or more specifically, from 4.6 F to 5.5 F. In another aspect, the catheter distal tip 43 has an ID 422 of 0.068 inches. In one or more embodiments, the flared end 42 is flared compared to the remainder of the catheter proximally (e.g., body of the catheter, or proximal shaft 41), with respect to either or both ID 422 and outer diameter (OD) 413.

In an aspect, the remainder of the VDAC proximally, including a proximal shaft 41, is just large enough in outer diameter 413 (e.g., about 0.054 inches in OD) and/or inner diameter 412 (e.g., about 0.044 inches in ID) to allow delivery of a microcatheter therein, and hence the delivery of a stent-retriever.

In an aspect, the flared end 42 of the VDAC 40 is large enough in outer diameter (OD) 423 (e.g., about 0.078-0.086 inches OD) and/or inner diameter 422 (e.g., about 4.6-5.5 F ID, or about 0.060-0.072 inches ID)) to capture the proximal aspect of clot with local aspiration.

In an aspect, the distal tip 42 of the VDAC 40 includes a narrowing, variable diameter portion 44, which is continuous with the proximal shaft 41 at its proximal end, and with the flared 43 at its distal end. Between the proximal end and the distal end of the variable diameter portion 44, the inner diameter 422 and/or the outer diameter 423 of the distal tip 42 are variable.

In an aspect, the proximal shaft 41 of the VDAC 40 according to an embodiment of the present disclosure has an outer diameter 413, which is smaller than the OD 423 of the flared end 42 of the VDAC 40, which allows true reversal of flow into the balloon guide catheter, so as to capture poorly integrated clot or loose fragments.

In an aspect, the OD 413 of the proximal shaft 41 of the VDAC 40 is small enough (e.g., about 0.050-0.060 inches OD) to allow flow of fluid around it (e.g., blood flow) when placed inside an 0.084 ID (or larger) balloon guide catheter.

In an aspect, the proximal shaft 41 of the VDAC 40 is robust enough (e.g., made of a material so as) to prevent collapse (of the shaft) under vacuum.

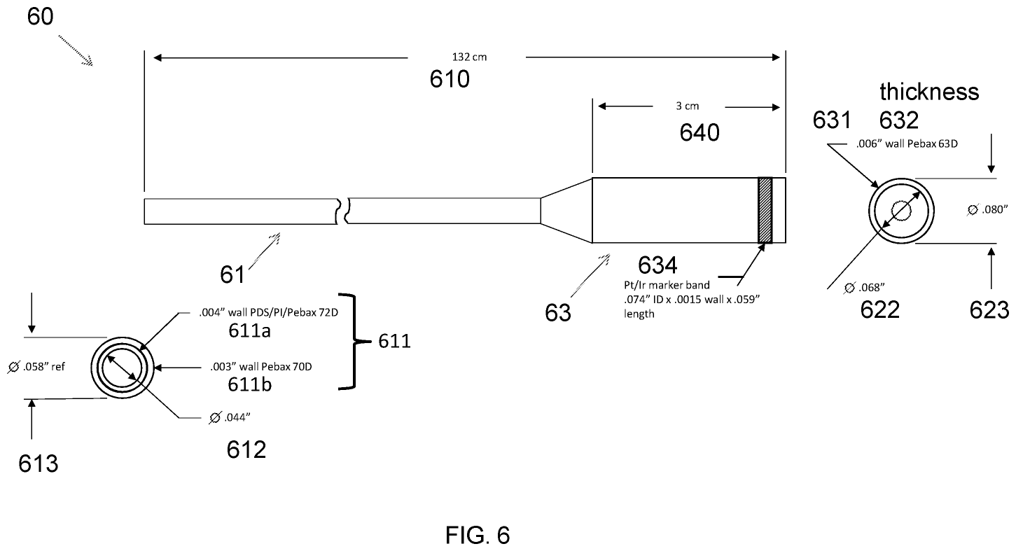

For example, referring to FIG. 6, the VDAC 60 according to an embodiment of the present disclosure has a total length 610 of about 132 cm. The flared end 63 of the VDAC 60 is about 3 cm in length 640, with an outer diameter 623 of about 0.080 inches, and an inner diameter 622 of about 0.068 inches. The wall 631 of the flared end 63 of the VDAC 60 may be about 0.006 inches in thickness 632. In an aspect, the flared end 63 of the VDAC 60 may be made of a polymer such as an elastomer suitable for medical application, such as Pebax® MED grades. In an aspect, the flared end 63 of the VDAC 60 may be made of Pebax® 63D (e.g., Pebax® 6333 SA 01 MED).

In an aspect, the flared end 63 of the VDAC 60 according to an embodiment of the present disclosure has a Pt/lr marker band 634 thereon (0.074 inches ID×0.0015 wall×0.059 inches length). The marker band 634 may indicate precise tip location of the VDAC 60.

In an aspect, the proximal shaft 61 of the VDAC 60 according to an embodiment of the present disclosure has an outer diameter 613 of about 0.058 inches, and an inner diameter 612 of about 0.044 inches. The wall 611 of the proximal shaft 61 of the VDAC 60 may be about 0.007 inches in total thickness. In an aspect, the proximal shaft 61 of the VDAC may be made of a polymer such as an elastomer suitable for medical application, such as Pebax® MED grades. In an aspect, the wall 611 of the proximal shaft 61 of the VDAC 60 may include an inside layer 611a made of 0.004″ wall PDS/PI/Pebax® 72D, and an outer layer 611b made of 0.003″ wall Pebax® 70D. In an aspect, the proximal shaft 61 of the VDAC 60 has an inside layer 611a that is made of sufficiently to highly lubricious material, such as PD Slick. In an aspect, the proximal shaft 61 of the VDAC 60 has a high collapse resistance due to a PDS/PI combination. In an aspect, the OD 613 of the proximal shaft 61 of the VDAC 60 typically provides about 0.013″-0.016″ annular space (not shown) for blood flow. The term, “annular space” is defined as the space between the outer wall of the proximal shaft of a VDAC and the inner wall of a balloon guide catheter (BGC, which typically ranges in ID from 0.084″ to 0.090″), when the VDAC is axially placed within the BGC. Its size is defined by the annular diameter of the space (i.e., the distance between the outer edge of the wall of the proximal shaft and the inner wall of the BCG). In an aspect, the proximal shaft 61 of the VDAC 60 exhibits good column strength for pushability For instance, despite the relatively small size and thin wall of the proximal shaft 61, it exhibits adequate rigidity to be easily advanced without kinking or crumpling.

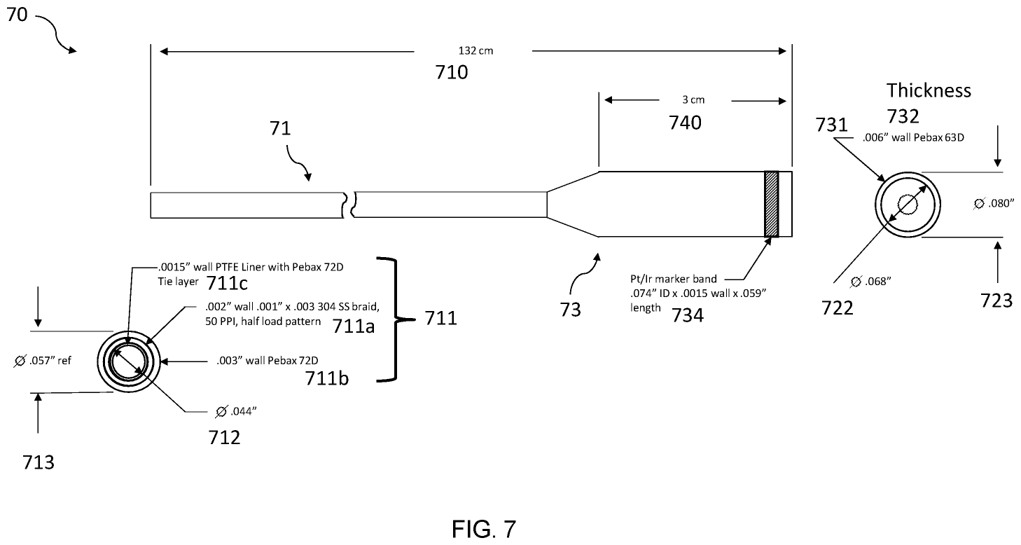

Referring to FIG. 7, the VDAC 70 according to another embodiment of the present disclosure has a total length 710 of about 132 cm. The flared end 73 of the VDAC 70 is about 3 cm in length 740, with an outer diameter 723 of about 0.080 inches, and an inner diameter 722 of about 0.068 inches. The wall 731 of the flared end 73 of the VDAC 70 may be about 0.006 inches in thickness 732. In an aspect, the flared end 73 of the VDAC 70 may be made of a polymer such as an elastomer suitable for medical application, such as Pebax® MED grades. In an aspect, the flared end 73 of the VDAC 70 may be made of Pebax® 63D (e.g., Pebax® 6333 SA 01 MED).

In an aspect, the flared end 73 of the VDAC 70 according to an embodiment of the present disclosure has a Pt/lr marker band 734 thereon (0.074 inches ID×0.0015 wall×0.059 inches length). The marker band 734 may indicate precise tip location of the VDAC 70.

In an aspect, the proximal shaft 71 of the VDAC 70 according to an embodiment of the present disclosure has an outer diameter 713 of about 0.057 inches, and an inner diameter 712 of about 0.044 inches. The wall 711 of the proximal shaft 71 of the VDAC 70 may be about 0.0065 inches in total thickness. In an aspect, the proximal shaft 71 of the VDAC 70 may be made of a polymer such as an elastomer suitable for medical application, such as Pebax® MED grades. In an aspect, the wall 711 of the proximal shaft 71 of the VDAC 70 may include an inside layer 711a made of 0.002″ wall 0.001″×0.003 304 SS braid, 50 PPI, half load pattern, a liner 711c that is sufficiently to fairly or highly lubricious, e.g., made of 0.015″ wall PTFE liner with Pebax® 72D tie layer, and an outer layer 711b made of 0.003″ wall Pebax® 72D. In an aspect, the proximal shaft 71 of the VDAC 70 has a wall 711 that is formed of a braid construction. In an aspect, the wall 711, and in turn the proximal shaft 71, of the VDAC 70 in accordance with an embodiment of the present disclosure is highly resistant to collapse due to the braid construction. In an aspect, the proximal shaft 71, and in turn the VDAC 70, provides good trackability due to the braid construction inside layer 711a that is made of sufficiently to highly lubricious material, such as PD Slick. In an aspect, the proximal shaft 71 of the VDAC 70 has a high collapse resistance due to a PDS/PI combination. In an aspect, the OD 713 of the proximal shaft 71 of the VDAC 70 typically provides about 0.0135″-0.0165″ annular space (not shown) for blood flow, depending on BGC ID. In an aspect, the proximal shaft 71 of the VDAC 70 also exhibits good column strength for pushability. The combination of good column strength and trackability, despite the relatively small size and thin wall of the proximal shaft 71, provides a balance between adequate rigidity (for easy advancement without kinking or crumpling) and navigability through tortuous anatomy.

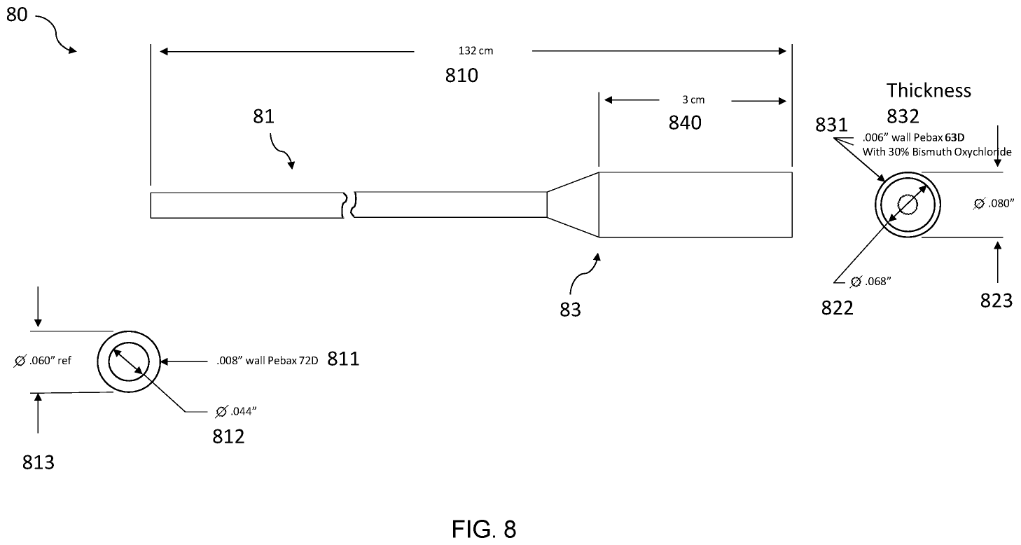

Referring to FIG. 8, the VDAC 80 according to another embodiment of the present disclosure has a total length 810 of about 132 cm. The flared end 83 of the VDAC 80 is about 3 cm in length 840, with an outer diameter 823 of about 0.080 inches, and an inner diameter 822 of about 0.068 inches. The wall 831 of the flared end 83 of the VDAC 80 may be about 0.006 inches in thickness 832. In an aspect, the flared end 83 of the VDAC 80 may be made of a polymer such as an elastomer suitable for medical application, such as Pebax® MED grades. In an aspect, the flared end 83 of the VDAC 80 may be made of Pebax® 63D (e.g., Pebax® 6333 SA 01 MED), with 30% Bismuth Oxychloride.

In an aspect, a portion or an entirety of the flared end 83 of the VDAC 80 may be radiopaque. In an aspect, the flared end 83 of the VDAC does not have a marker band thereon.

In an aspect, the proximal shaft 81 of the VDAC 80 according to an embodiment of the present disclosure has an outer diameter 813 of about 0.060 inches, and an inner diameter 812 of about 0.044 inches. The wall 811 of the proximal shaft 81 of the VDAC 80 may be about 0.008 inches in total thickness. In an aspect, the proximal shaft 81 of the VDAC 80 may be made of a polymer such as an elastomer suitable for medical application, such as Pebax® MED grades. In an aspect, the wall 811 of the proximal shaft 81 of the VDAC 80 is made of 0.008″ wall Pebax® 72D. In an aspect, the OD 813 of the proximal shaft 81 of the VDAC 80 typically provides about 0.012″-0.015″ annular space (not shown) for blood flow, depending on BGC ID. In an aspect, the proximal shaft 81 of the VDAC 80 exhibits good column strength for pushability. For instance, despite the relatively small size and thin wall of the proximal shaft 81, it exhibits adequate rigidity to be easily advanced without kinking or crumpling.

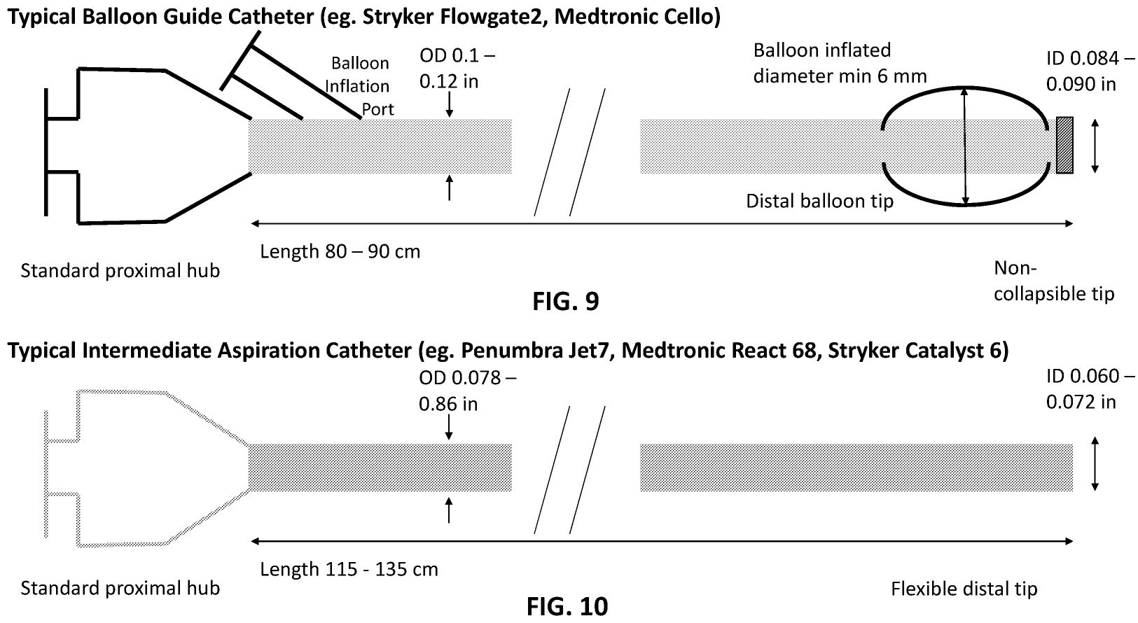

FIG. 9-12 respectively show schematic illustrations of a typical balloon guide catheter, a typical intermediate aspiration catheter, a typical delivery microcatheter, and a variable diameter aspiration catheter in accordance with certain embodiments of the present disclosure, each shown as attached to a standard proximal, luer-lock type hub.

Referring to FIG. 9, a schematic of a typical balloon guide catheter 100 is illustrated, shown as attached to a standard proximal hub at its proximal end. Non-limiting examples of the typical balloon guide catheter 100 having a balloon tip 110 that may be used with the VDAC 80 according to one more embodiments of the present disclosure include Stryker Flowgate2, Medtronic Cello, etc., which may securely engage a standard luer lock type hub on its proximal end. In an aspect, a typical balloon guide catheter 100 has a balloon inflation port (and relatively tiny lumen) for balloon inflation. In some aspects, a balloon guide catheter 100 has one or more of the following dimensions: OD of about 0.1-0.12 inches, ID of about 0.084-0.090 inches, length of about 80-90 cm, and a balloon with an inflated diameter of at least about 6 millimeters (mm). In an aspect, the balloon guide catheter has sufficient resistance to collapse when suction (vacuum) is applied such that flow reversal is possible when a VDAC 80 according to one or more embodiments of the present disclosure is used with the typical balloon guide catheter 100.

Referring to FIG. 10, a schematic of a typical conventional intermediate aspiration catheter 200 is illustrated, shown as attached to a standard proximal hub at its proximal end. Non-limiting examples of the typical conventional intermediate catheter 100 include Penumbra Jet7, Medtronic React 68, Catalyst 6, etc., which may securely engage a standard luer lock type hub on its proximal end. In some aspects, a conventional intermediate aspiration catheter has one or more of the following dimensions: OD of about 0.078-0.86 inches, ID of about 0.060-0.072 inches, length of about 115-135 cm, and a flexible distal tip.

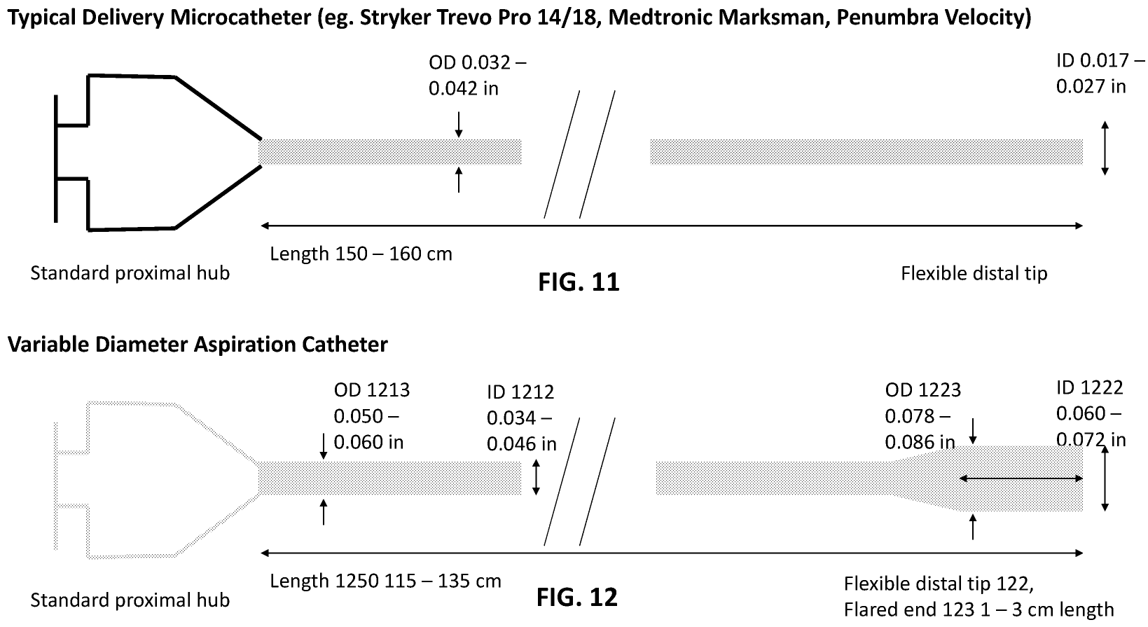

Referring to FIG. 11, a schematic of a typical delivery microcatheter 300 is illustrated, shown as attached to a standard proximal hub at its proximal end. Non-limiting examples of the typical microcatheter 300 that may be used with the VDAC 80 according to one or more embodiments of the present disclosure include Stryker Trevo Pro 14/18, Medtronic Marksman, Penumbra Velocity, etc., which may securely engage a standard luer lock type hub on its proximal end. In some aspects, a microcatheter 300 has one or more of the following dimensions: OD of about 0.032-0.042 inches, ID of about 0.017-0.027 inches, length of about 150-160 cm, and a flexible distal tip.

Referring to FIG. 12, a schematic of a VDAC 80 according to one or more embodiments of the present disclosure is illustrated, shown as attached to a standard proximal hub at its proximal end. In some aspects, VDAC 80 including a distal tip 122 (42: FIG. 4) has one or more of the following dimensions: shaft OD 1213 (413) of about 0.050-0.060 inches, shaft ID 1212 (412) of about 0.034-0.046 inches, length 1250 of about 115-135 cm, a tip OD 1223 (423) of about 0.078-0.086 inches, a tip ID 1222 (422) of about 0.060-0.072 inches, a flared end 123 (43) of about 1-3 cm in length. In an aspect, the distal tip 122 is flexible.

Technique #5—Thrombectomy Technique with VDAC

In an aspect, the thrombectomy technique with VDAC is set up the same as TRAP described above (Technique #4).

In an aspect, the stent-retriever is deployed across the clot and allowed to integrate.

In an aspect, the flared distal end of the VDAC is similar in size to typical intermediate aspiration catheters, and is large enough to capture the proximal aspect of clot with local suction.

In an aspect, the balloon guide is inflated to arrest flow in the ICA.

In an aspect, unlike TRAP with a standard intermediate aspiration catheter, TRAP with VDAC allows “full flow control” with the addition of flow reversal in the ICA when suction is also applied to the balloon guide. The narrow proximal end of the VDAC is small enough to allow flow around it/within the balloon guide catheter.

In an aspect, the stent-retriever and VDAC are removed as a single unit, without fully retracting the retriever into the VDAC.

In an aspect, similar to TRAP, TRAP with VDAC allows control of both proximal and distal ends of the thrombus, decreasing the likelihood of loss of dislodged clot and clot fragmentation. Concomitant flow reversal with the balloon guide and VDAC allows capture of any loose clot fragments, theoretically enhancing the first pass effect.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language mans that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A thrombectomy catheter comprising:
a distal tip having:
a tip inner diameter (ID);
a tip outer diameter (OD);
a tip distal end;
a tip proximal end;
a flared end towards the tip distal end having a flared end ID and a flared end OD; and
a variable diameter portion towards the tip proximal end having a variable outer diameter (OD);
a proximal shaft having:
a shaft inner diameter (ID);
a shaft outer diameter (OD);
a shaft proximal end; and
a shaft distal end; the shaft distal end being continuous with the tip proximal end.

2-25. (canceled)
